(19) United States
(12) Patent Application Publication
(10) Pub. No.: US 2018/0161369 A1
(43) Pub. Date: Jun. 14, 2018

(54) COMPOSITIONS FOR CELLULAR IMMUNOTHERAPY

(71) Applicant: Fred Hutchinson Cancer Research Center, Seattle, WA (US)

(72) Inventor: Stanley R. RIDDELL, Sammamish, WA (US)

(21) Appl. No.: 15/577,280

(22) PCT Filed: May 27, 2016
(86) PCT No.: PCT/US2016/034860

§ 371 Date: Nov. 27, 2017

Related U.S. Application Data
(60) Provisional application No. 62/168,675, filed on May 29, 2015.

(51) Int. Cl. A61K 35/17; C07K 14/705; C12N 5/0783; A61P 35/00; C07K 16/28

(52) U.S. Cl. CPC A61K 35/17; C07K 2317/80; C12N 5/0636; A61P 35/00; C07K 14/70575; C07K 14/70578; C07K 14/70596; C07K 16/2803; C12N 2510/00; C07K 2317/622; C07K 2319/70; C07K 2317/24; C07K 2317/565; C07K 2317/732; C07K 14/70521

(57) ABSTRACT

The present disclosure relates to compositions and methods for using cells having cellular immunotherapies comprising a composition of chimeric antigen receptor (CAR)-modified CD4+ T cells and a composition of CD8+ T cells modified with a distinct CAR in that the intracellular signaling component of the each CAR is different, wherein the modified CD4+ and CD8+ T cells have enhanced helper and effector functions, respectively, and together more effectively augment the immune response. Such cellular immunotherapies are useful in treating disease, such as cancer.

### BACKGROUND

Adoptive immunotherapy using chimeric antigen receptors (CARs) provides a promising approach for cancer treatment. CARs can endow T cells with MHC independent specificity for cell surface antigens using antigen-binding domains, typically single-chain variable fragments (scFvs), linked to T cell signaling domains (Sadelain et al., 2013, Cancer Discovery 3:388-398). Preclinical and clinical studies have demonstrated potent anti-tumor activity of CD19 specific CAR T cells against B cell malignancies (Lee et al., 2015, Lancet 385:517-528; Brentjens et al., 2013, Sci. Transl. Med. 5:177ra138; Kochenderfer et al., 2012, Blood 119:2709-2720; Porter et al., 2011, N. Engl. J. Med. 365:725-733).

However, CAR therapy against solid tumors has shown less anti-tumor efficacy (Kershaw et al., 2006, Clin. Cancer Res. 12:6106-6115; Park et al., 2007, Mol. Ther. 15:825-833; Pule et al., 2008, Nat. Med. 14:1264-1270; Lamers et al., 2013, Mol. Ther. 21:904-912; Louis et al., 2011, Blood 118:6050-6056). The variable response may be due in part to challenges particular to solid tumors compared with B-cell malignancies. Such challenges may include lower sensitivity to T cell mediated cytotoxicity, immunosuppressive tumor microenvironment that presents immunosuppressive mechanisms, and the need to identify target antigens with appropriate “favorable” expression profile similar to that of CD19. Variation in CAR functionality may also impact efficacy, particularly in solid tumors. Efficient exposure, e.g., by way of expansion and/or persistence, of adoptively transferred T cells in vivo can be important to effective anti-tumor responses (Robbins et al., 2004, J. Immunol. 173:7125-7130; Kowolik et al., 2006, Cancer Res. 66:10995-11004; Milone et al., 2009, Mol. Ther. 17:1453-1464; Haso et al., 2013, Blood 121:1165-1174). There is a need for improved approaches to chimeric antigen receptor design to promote CAR potency.

For example, there is a need for improved chimeric antigen receptor design approaches that provide enhanced in vivo expansion, survival, and response of adoptively transferred CAR-modified T cells, particularly in solid tumors. Provided by the present disclosure are approaches and embodiments addressing such needs, and further providing other related advantages.

### DETAILED DESCRIPTION

In certain aspects, the present disclosure provides distinct modified T cell compositions that form a multi-component preparation useful in immunotherapy, such as adoptive immunotherapy. For example, the present disclosure provides cellular immunotherapies comprising a composition of CD4+ T cells genetically modified (engineered) to contain a first chimeric antigen receptor (CAR) and a composition of CD8+ T cells genetically modified (engineered) to contain a second CAR, in which the first and second CARs have certain different features. In some aspects, each of the first and second CARs specifically bind to an antigen (which generally is the same antigen and may be by way of the same or a different antigenic epitope) and each CAR contains an intracellular costimulatory domain, provided that the intracellular costimulatory domain of the first CAR (first intracellular costimulatory domain) is distinct from the intracellular costimulatory domain of the second CAR (second intracellular costimulatory domain). Generally, (a) the CD4+ T cells do not contain the second CAR, (b) the CD4+ T cells do not contain a CAR comprising the second intracellular costimulatory domain, (c) the CD8+ T cells do not contain the first CAR, (d) the CD8+ T cells do not contain a CAR comprising the first intracellular costimulatory domain, or (e) any combination of (a)-(d).

Among the provided adoptive cellular immunotherapies are those designed to provide a composition enriched for a CD4+ subpopulation of T cells containing a specific CAR and a composition enriched for a CD8+ subpopulation of T cells containing a different specific CAR, wherein each distinct CAR has a specific intracellular costimulatory domain. In some aspects, the respective costimulatory domains are stimulatory or signaling regions present in costimulatory receptor(s) that are endogenous to the particular T cell subpopulation (CD4+ or CD8+), such as a costimulatory receptor that in a natural setting is upregulated upon certain stimulatory conditions, in some cases to a greater degree or more rapidly as compared to the other subpopulation, and/or in a natural setting can effectively enhance the effector function or a particular desired effector function (such as secretion of a particular cytokine(s) or cell killing or survival) of that particular T cell subpopulation. As such, the combination of enriched T cell populations in the composition (each with a CAR designed for particular efficacy in the respective population or desired effector function thereof) produces a more potent and prolonged immune response. The use of such modified CD4+ and CD8+ cell populations together can provide an advantage in diseases, for example, wherein the number of antigen targets is low or difficult to access, such as solid tumors.

Without wishing to be bound by theory, for example, a CD4+ T cell subpopulation having a CAR that contains an intracellular costimulatory domain that is present in a costimulatory molecule that is upregulated in response to certain stimuli (or upregulated more rapidly as compared to in CD8+ cells) when present endogenously in CD4+ T cells, or that is known to promote a function (such as secretion of a particular cytokine or cytokine profile, e.g., Th1 cytokines, such as IL-2) desired of the CD4+ cells in promoting a particular help to promote desired effects or impacts on cytotoxic T cells in the context of cellular immunotherapy. Such costimulatory molecules may include, for example, CD28 or ICOS. In some aspects, the use of CARs with such tailored costimulatory domains in the CD4+-enriched population will result in CAR-modified CD4+ T cells, e.g., in combination with a CD8+-enriched population similarly tailored for specific efficacy in CD8+ cells, with enhanced helper T cell function or enhanced function of a particular type. In some aspects, this feature in turn increases desired in vivo effects of the CD8+ T cells of the composition, such as enhancing proliferation, persistence, homing, access to tumor microenvironment, and anti-tumor reactivity or efficacy of CD8+ CAR-modified T cells.

Similarly, a CD8+ T cell subpopulation having a CAR that contains an intracellular costimulatory domain present in a costimulatory molecule that is upregulated in response to certain stimuli (or upregulated more rapidly as compared to in CD4+ cells) when present endogenously in CD8+ T cells, or that is known to promote a function or outcome (such as enhanced survival, reduced inhibition by tumor microenvironment, or increased efficacy) desired in particular of the CD8+ T cells in the composition, and in some aspects which may not be particularly desirable or necessary in CD4+ cells in the composition. Such costimulatory molecules may include, for example, CD27, CD40L or 4-1BB. In some embodiments, such CD8+ T cells, e.g., when used in combination with tailored CD4+ T cells with CARs having distinct costimulatory domains, can have an augmented immune response (e.g., more cytotoxic effects, increased expansion and/or persistence, resistance to negative regulation by a tumor microenvironment, access to solid tumor, and/or homing).

Provided are such compositions with improved efficacy, as compared to alternative compositions for adoptive immunotherapy including both CD4+ and CD8+ T cells in which (1) T cells of different subpopulations (e.g., CD4+ and CD8+ enriched populations) contain CARs having the same costimulatory domain(s), rather than distinct costimulatory domains, and/or in which (2) T cells in the composition express a CAR which itself has multiple costimulatory domains (such as one domain tailored towards promoting an effect that is particularly advantageous for CD4+ cells and another tailored toward promoting an effect that is particularly advantageous in CD8+ cells), e.g., so-called “third generation” CARs.

As to (1) and as described herein, the different subpopulations in a combined T cell immunotherapy composition serve different functions and in turn can particularly benefit from different costimulatory signals, for example, a signal particularly good at promoting a desired cytokine profile in CD4+ cells, which in turn will deliver optimal help to the CD8+ cells, or a signal particularly tailored to promote longevity, expansion, or effector function in the CD8+ population. Thus, using the same CAR for each subpopulation can in some contexts not be the optimal choice.

As to (2), a potential solution to this concern may be to use a so-called third generation CAR in each population, with the goal of providing each subpopulation with the signal that is particularly advantageous and/or desired for that cell population.

Nonetheless, use of CARs with multiple costimulatory domains in some embodiments may not be advantageous and in some contexts may be less optimal than a single costimulatory domain. Without being bound by theory, delivering a signal via a given costimulatory domain can involve the activation of multiple different signaling cascades and/or promote a range of different effects. Thus, in addition to a particular effect or effects intended (e.g., IL-2 secretion), there may be a range of different impacts from signaling through a particular costimulatory domain. If the intended effect(s) are not particularly advantageous for one sub-population or are not as advantageous as they are in another sub-population, it can be that less desirable effects that are also promoted could outweigh the benefit of including that costimulatory domain in cells of that subpopulation.

With embodiments provided herein, CARs in the different subpopulations are specifically designed, with costimulatory domain(s) designed (e.g., harnessing endogenous signals known to be activated in the context of desired outcomes in the natural setting for each respective subpopulation) to promote optimal or desired effects for that cell population in particular, and without the inclusion of any costimulatory or signaling domain(s) that would advantage another subpopulation but provide no advantage or no overall advantage to the subpopulation in question.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means ±20% of the indicated range, value, or structure, unless otherwise indicated. The term “consisting essentially of” limits the scope of a claim to the specified materials or steps, or to those that do not materially affect the basic and novel characteristics of the claimed invention. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

As used herein, the term “adoptive cellular immunotherapy” or “adoptive immunotherapy” refers to the administration of naturally occurring or genetically engineered, disease antigen-specific immune cells (e.g., T cells). Adoptive cellular immunotherapy may be autologous (immune cells are from the recipient), allogeneic (immune cells are from a donor of the same species) or syngeneic (immune cells are from a donor genetically identical to the recipient).

As used herein, “T cells” or “T lymphocytes” are from any mammal, including primates, dogs, or horses, preferably humans. In some embodiments, T cells are autologous, allogeneic, or syngeneic.

As used herein, the term “CD4+ T cell” or “CD4+ T lymphocyte” refers to a T cell that expresses CD4 on the surface thereof. CD4+ T cells include naive CD4+ T cells (CD4+ T_{N}), helper T cells (CD4+ T_{H}), memory stem CD4+ T cells (CD4+ T_{MSC}), central memory CD4+ T cells (CD4+ T_{CM}), effector memory CD4+ T cells (CD4+T_{EM}), effector CD4+ T cells (CD4+ T_{E}), or any combination thereof.

As used herein, the term “naive CD4+ T cell” refers to a non-antigen experienced CD4+ T cell that expresses CD62L and CD45RA, and does not express or has decreased expression of CD45RO as compared to central memory CD4+ cells. In some embodiments, naive CD4+ T cells are characterized by the expression of phenotypic markers of naïve T cells including CD62L, CCR7, CD28, CD3, CD127, and CD45RA.

As used herein, the term “helper T cells” refers to an activated, as opposed to naive, T lymphocyte that expresses CD4 on its surface. Naive CD4+ T cells become helper T cells following activation due to interaction with a MHC class II-restricted peptide antigen complex and co-stimulation via CD28. CD4+ helper T cells include both effector CD4+ T cells and memory CD4+ T cells.

“CD4+ T effector cells” (CD4+ T_{E}) do not express or have decreased expression of CD62L, CCR7, CD28, and are positive for expression of cytokines specific for each CD4+ T effector cell subtype (e.g., IL-2, IFN-γ, TNF-α or TNF-β for TH1 CD4+ T effector cells; IL-4, IL-5, IL-9, IL-10 or IL-13 for TH2 CD4+ T effector cells) as compared to central memory CD4+ T cells. Other CD4+ T effector cell subtypes include THO CD4+ T effector cells, TH9 CD4+ T effector cells, TH17 CD4+ T effector cells, Treg CD4+ T effector cells, and Tfh CD4+ T effector cells.

As used herein, the term “TH1 CD4+ T effector cells” or “TH1 helper T cells” refer to CD4+ T effector cells that produce pro-inflammatory cytokines, also known as TH1 cytokines. A TH1 cytokine may be IL-2, IFN-γ, TNF-α, TNF-β, GM-CSF, or any combination thereof. TH1 CD4+ T effector cells promote cell-mediated immunity.

As used herein, the term “TH2 CD4+ T effector cells” or “TH2 helper T cells” refer to CD4+ T effector cells that produce TH2 cytokines. A TH2 cytokine may be IL-4, IL-5, IL-6, IL-9, IL-10, IL-13, IL-17E (IL-25), or any combination thereof. TH2 CD4+ T effector cells promote humoral immunity.

As used herein, the term “memory CD4+ T cells” (CD4+ T_{M}) refer to antigen experienced CD4+ T cells that provide long lasting immunity. Memory CD4+ T cells are long lived, inactive CD4+ T cells that are able to rapidly acquire effector functions upon antigen re-challenge. Memory CD4+ T cells include memory stem CD4+ T cells (CD4+ T_{MSC}), central memory CD4+ T cells (CD4+ T_{CM}), and effector memory CD4+ T cells (CD4+ T_{EM}).

As used herein, “central memory CD4+ T cell” (CD4+ T_{CM}) refers to an antigen experienced helper T cell that expresses CD62L and CD45RO on the surface thereof, and does not express or has decreased expression of CD45RA as compared to naive CD4+ T cells. Central memory CD4+ T cells have a longer lifespan than CD4+ T_{E} cells and CD4+ T_{EM} cells and can differentiate into CD4+ T_{EM} cells following antigenic challenge. In some embodiments, central memory CD4+ T cells are positive for expression CD62L, CCR7, CD28, CD127, CD45RO, and CD95, and have decreased expression of CD54RA as compared to naive CD4+ T cells.

As used herein, “effector memory CD4+ T cell” (CD4+ T_{EM}) refers to an antigen experienced helper T cell that does not express or has decreased expression of CD62L on the surface thereof as compared to central memory cells, and does not express or has decreased expression of CD45RA as compared to naive CD4+ T cells. Effector memory CD4+ T cells are terminally differentiated and acquire effector function immediately after re-stimulation by the same antigen. In some embodiments, effector memory CD4+ T cells are negative for expression CD62L, CCR7, CD28,CD45RA, and are positive for CD127 as compared to naive CD4+ cells or central memory CD4+ T cells.

As used herein, “memory stem CD4+ T cell” (CD4+ T_{SCM}) refers to an antigen experienced helper T cell that expresses CD45RA, CD62L, CD95, and CD122. T_{SCM} cells possess memory T cell capability of rapid acquisition of effector function following antigen re-challenge, but have enhanced stem cell-like qualities compared to T_{CM} cells. T_{SCM} cells can generate central memory, effector memory, and effector T cell subsets.

As used herein, the term “CD8+ T cell” or “CD8+ T lymphocyte” refers to a T cell that expresses CD8 on the surface thereof. CD8+ T cells include naive CD8+ T cells, cytotoxic T lymphocytes (CTLs), memory stem CD8+ T cell (CD8+ T_{MSC}), central memory CD8+ T cells (CD8+ T_{CM}), effector memory CD8+ T cells (CD8+ T_{EM}), effector CD8+ T cells (T_{E}), or any combination thereof.

As used herein, the term “naïve CD8+ T cell” refers to a non-antigen experienced CD8+ T cell that expresses CD62L and CD45RA, and does not express or has decreased expression of CD45RO as compared to central memory CD4+ cells. In some embodiments, naive CD8+ T cells are characterized by the expression of phenotypic markers of naive T cells including CD62L, CCR7, CD28, CD3, CD127, and CD45RA.

As used herein, the term “cytotoxic T cell,” also known as T_{C}, cytotoxic T lymphocyte, CTL, killer T cell, or cytolytic T cell, refers to an activated, as opposed to naive, T lymphocyte that expresses CD8 on its surface. Naïve CD8+ T cells become CTLs following activation by interacting with a MEW class I-restricted peptide antigen complex and co-stimulation via CD28. CD8+ CTLs include both effector CD8+ T cells and memory CD8+ T cells.

As used herein, the term “effector CD8+ T cells” (CD8+ T_{E}) refer to antigen experienced CTLs that do not express or have decreased expression of CD62L, CCR7, CD28, and are positive for granzyme B and perforin as compared to central memory CD8+ T cells. Effector CD8+ T cells possess cytotoxic activity towards cells expressing the target antigen and are short lived as compared to CD8+ T_{M} cells.

As used herein, the term “memory CD8+ T cells” (CD8+ T_{M}) are antigen experienced CD8+ T cells that provide long lasting immunity. Memory CD8+ T cells are long lived, inactive CD8+ T cells that are able to rapidly acquire effector functions upon antigen re-challenge. Memory CD8+ T cells include memory stem CD8+ T cells (T_{MSC}), central memory (T_{CM}) CD8+ T cells, and effector memory CD8+ T cells (T_{EM}).

As used herein, “central memory CD8+ T cell” (CD8+ T_{CM}) refers to an antigen experienced CTL that expresses CD62L and CD45RO on the surface thereof, and does not express or has decreased expression of CD45RA as compared to naive CD8+ T cells. Central memory CD8+ T cells have a longer lifespan than CD8+ T_{E} and CD8+ T_{EM} cells and can differentiate into effector memory CD8+ T cells following antigenic challenge. In some embodiments, central memory CD8+ T cells are positive for expression CD62L, CCR7, CD28, CD127, CD45RO, and CD95, and have decreased expression of CD54RA as compared to naive CD8+ T cells.

As used herein, “effector memory CD8+ T cell” (CD8+ T_{EM}) refers to an antigen experienced CTL that does not express or has decreased expression of CD62L on the surface thereof as compared to central memory cells, and does not express or has decreased expression of CD45RA as compared to naive CD8+ T cells. Effector memory CD8+ T cells are terminally differentiated and acquire effector function immediately after re-stimulation by the same antigen. In some embodiments, effector memory CD8+ T cells are negative for expression CD62L, CCR7, CD28, CD45RA, and are positive for CD127 as compared to naive cells or central memory cells.

As used herein, “memory stem CD8+ T cell” (T_{SCM}) refers to an antigen experienced CTL that expresses CD45RA, CD62L, CD95, and CD122. T_{SCM} cells possess memory T cell capability of rapid acquisition of effector function following antigen re-challenge, but have enhanced stem cell-like qualities compared to T_{CM} cells. T_{SCM} cells can generate central memory, effector memory, and effector T cell subsets.

As used herein, the term “chimeric antigen receptor” (CAR) refers to a fusion protein engineered to contain two or more naturally-occurring amino acid sequences linked together in a way that does not occur naturally or does not occur naturally in a host cell, which fusion protein can function as a receptor when present on the surface of a cell and comprises an extracellular antigen binding domain specific for an antigen, a hydrophobic portion or transmembrane domain, and an intracellular signaling component that is at minimum capable of activating or stimulating a T cell. An intracellular signaling component may be a T cell or other receptor (e.g., TNFR superfamily member) or portion thereof, such as an intracellular activation domain (e.g., an immunoreceptor tyrosine-based activation motif (ITAM)-containing T cell activating motif), an intracellular costimulatory domain, or both. A hydrophobic portion or transmembrane domain is disposed between the extracellular antigen binding domain and the intracellular signaling component, which transverses and anchors the CAR in a host cell membrane (e.g., T cell). A chimeric antigen receptor may further comprise an extracellular spacer domain connecting the hydrophobic portion or transmembrane domain and the extracellular antigen binding domain.

Exemplary CARs may have two or more portions from the same protein linked in a way not normally found in a cell, or a CAR may have portions from two, three, four, five or more different proteins linked in a way not normally found in a cell. Furthermore, CARs can be in the form of first, second or third generation CARs. For example, a first generation CAR generally may have a single intracellular signaling domain providing an activating signal (e.g., intracellular signaling domain of CD3 or FcyRT or other ITAM-containing domain). Second generation CARs further include an intracellular costimulatory domain (e.g., a costimulatory domain from an endogenous T cell costimulatory receptor, such as CD28, 4-1BB, or ICOS). Third generation CARs further include a second costimulatory domain. In some embodiments, the chimeric antigen receptors of this disclosure are not third generation CARs and/or provide advantages as compared to available third-generation CARs or compositions containing the same. In some embodiments, the provided compositions include cells with third-generation CARs, but generally with one set of costimulatory domains on a population enriched for CD4+ or other subpopulation of T cells on the one hand and a different set of costimulatory domains on a population enriched for CD8+ cells or other subpopulation on the other hand.

A CAR can be encoded by a nucleic acid molecule wherein a first nucleotide sequence encoding one protein or portion thereof is appended in frame with a second nucleotide sequence encoding one or more different proteins or a portion thereof, and optionally the first and second nucleotide sequences are separated by nucleotides that encode a linker, spacer or junction amino acid(s) (natural or non-natural). In certain embodiments, a nucleic acid molecule encoding a CAR is introduced into a host cell and expressed.

As used herein, the term “intracellular costimulatory domain” refers to an intracellular signaling domain or functional portion thereof present on a co-stimulatory molecule (e.g., CD28, 4-1BB, TNFR superfamily member), which, when activated in addition to a primary or classic (e.g., ITAM-driven) activation signal (provided by, for instance, a CD3 chain of the TCR/CD3 complex), promotes or enhances a T cell response, such as T cell activation, cytokine production, proliferation, differentiation, survival, effector function, or combinations thereof. Examples of intracellular co-stimulatory domains include CD27, CD28, 4-1BB (CD137), OX40 (CD134), CD30, CD4OL, CD226, DR3, GITR, HVEM, ICOS (CD278), lymphocyte function-associated antigen-1 (LFA-1), CD2, CD7, LIGHT, NKG2C, SLAM, and TIM1. The intracellular costimulatory domain may be any portion of such a costimulatory molecule that retains signaling activity.

An “ITAM-containing T cell activating motif” refers to an intracellular signaling domain or portion thereof (which is naturally or endogenously present on an immune cell receptor or a cell surface marker and contains at least one immunoreceptor tyrosine-based activation motif (ITAM)). ITAMs are generally known to be capable of initiating T cell activation signaling following ligand engagement. ITAM-containing T cell activating motifs include, for example, intracellular signaling domains of CD3γ, CD3δ, CD3ϵ, CD3ζ and gamma chain of FcϵRI or FcγRI.

As used herein, the term “antigen” refers to any substance that provokes or is capable of provoking an immune response. Such an immune response may involve antibody production, cell-mediated immunity, or both. An antigen can be generated, synthesized, present in or derived from a biological sample, such as a tissue sample, a tumor sample, a cell or a biological fluid (e.g., blood or serum). In some embodiments, an antigen is a peptide or a peptide complexed with an MHC or HLA complex. For example, an antigenic peptide may be an HLA Class I peptide, an HLA Class II peptide, or an HLA Class II peptide having an embedded HLA Class I peptide.

As used herein, an “antigen binding domain” refers to a domain, such as a domain of a polypeptide that specifically binds to a target antigen. An antigen binding domain may be from a natural antibody, synthetic antibody construct, or a fragment thereof. For example, an antigen binding domain may be a full length heavy chain, Fab fragment, Fab′, F(ab′)₂, VH region, VL region, a domain antibody (dAb), a camelid antibody (VHH), a complementary determining region (CDR), or single chain Fv fragment (scFv). Other examples of antigen binding domains include antigen-binding portions of (or full-length) T cell receptors, such as single chain T cell receptors (scTCRs), extracellular domains of receptors, ligands, tumor binding proteins/peptides, and cytokines.

As used herein, “specifically binds” or “specific for” refers to an association or union of a binding protein (e.g., CAR) or a binding domain (or fusion protein thereof) to a target (molecule or complex) with an affinity or Kₐ (i.e., an equilibrium association constant of a particular binding interaction with units of 1/M) equal to or greater than 10⁵ M⁻¹ (which equals the ratio of the on-rate [kₒₙ] to the off-rate [k_{off}] for this association reaction), while not significantly associating or uniting with any other molecules or components in a sample. Binding proteins or binding domains (or fusion proteins thereof) may be classified as “high affinity” binding proteins or binding domains (or fusion proteins thereof) or as “low affinity” binding proteins or binding domains (or fusion proteins thereof). “High affinity” binding proteins or binding domains refer to those binding proteins or binding domains having a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low affinity” binding proteins or binding domains refer to those binding proteins or binding domains having a Kₐ of up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M).

As used herein, “enriched” and “depleted” with respect to amounts of cell types in a mixture refers to a mixture of the cells subjected to a process or step that results in an increase in the number of the “enriched” type, a decrease in the number of the “depleted” cells, or both. Thus, depending upon the source of the original population of cells subjected to the enriching process, a mixture or composition may contain 30%, 35%, 40%, 45%, 50%, 55%, 60%, 65%, 70%, 75%, 80%, 85%, 90%, 91%, 92%, 93%, 94%, 95%, 96%, 97%, 98%, or 99% or more (in number or count) of the “enriched” cells. Cells subjected to a depleting process can result in a mixture or composition containing 50%, 45%, 40%, 35%, 30%, 25%, 20%, 15%, 10%, 9%, 8%, 7%, 6%, 5%, 4%, 3%, 2%, or 1% percent or less (in number or count) of the “depleted” cells. In certain embodiments, amounts of a certain cell type in a mixture will be enriched and amounts of a different cell type will be depleted, such as enriching for CD4⁺ cells while depleting CD8⁺ cells, or enriching for CD62L⁺ cells while depleting CD62L⁻ cells, or combinations thereof.

As used herein “endogenous” refers to any material that is normally present or produced inside a host organism, cell, tissue or system.

As used herein, the term “exogenous” refers to any material introduced from or produced outside an organism, cell, tissue or system.

“MHC-peptide tetramer staining” refers to an assay used to detect antigen-specific T cells, which features a tetramer of MHC molecules, each comprising a peptide having an amino acid sequence that is cognate (e.g., identical or related) to at least one antigen (e.g., tumor-associated antigen), wherein the complex is capable of binding T cell receptors specific for the cognate antigen. Each of the MHC molecules may be tagged with a biotin molecule. Biotinylated MHC/peptides are tetramerized by the addition of streptavidin, which can be fluorescently labeled. The tetramer may be detected by flow cytometry via the fluorescent label. In certain embodiments, an MHC-peptide tetramer assay is used to detect or select enhanced affinity TCRs of the instant disclosure.

Levels of cytokines may be determined according to methods described herein and practiced in the art, including for example, ELISA, ELISPOT, intracellular cytokine staining, and flow cytometry and combinations thereof (e.g., intracellular cytokine staining and flow cytometry). Immune cell proliferation and clonal expansion resulting from an antigen-specific elicitation or stimulation of an immune response may be determined by isolating lymphocytes, such as circulating lymphocytes in samples of peripheral blood cells or cells from lymph nodes, stimulating the cells with antigen, and measuring cytokine production, cell proliferation and/or cell viability, such as by incorporation of tritiated thymidine or non-radioactive assays, such as MTT assays and the like. The effect of an immunogen described herein on the balance between a Th1 immune response and a Th2 immune response may be examined, for example, by determining levels of Th1 cytokines, such as IFN-γ, IL-12, IL-2, and TNF-β, and Type 2 cytokines, such as IL-4, IL-5, IL-9, IL-10, and IL-13.

### Chimeric Antigen Receptors

The CD4+ and CD8+ T cells for use as adoptive immunotherapy compositions described herein are genetically engineered to contain chimeric antigen receptors. Chimeric antigen receptors comprise an antigen binding domain, an optional extracellular spacer domain, a hydrophobic portion or transmembrane domain, and an intracellular signaling component, such as an intracellular activation domain (e.g., an immunoreceptor tyrosine-based activation motif (ITAM)-containing T cell activating motif), an intracellular costimulatory domain, or both. In particular embodiments, an intracellular signaling component of a CAR has an ITAM-containing T cell activating motif (e.g., CD3) and an intracellular costimulatory domain (e.g., CD27, CD28). In certain embodiments, a CAR is synthesized as a single polypeptide chain or is encoded by a nucleic acid molecule as a single chain polypeptide.

An antigen binding domain suitable for use in a CAR of the present disclosure can be any antigen-binding polypeptide. An antigen binding domain may comprise a natural antibody, synthetic or recombinant antibody construct, or a binding fragment thereof. For example, an antigen binding domain may comprise a full length heavy chain, Fab fragment, Fab′, F(ab′)₂, variable heavy chain domain (VH domain), variable light chain domain (VL domain), domain antibody (dAb), single domain camelid antibody (VHH), complementary determining region (CDR), or single chain antibody fragment (scFv). Other examples of antigen binding domains include single chain T cell receptors (scTCRs), extracellular domains of receptors, ligands for cell surface receptors/molecules, tumor binding proteins/peptides, and cytokines. In some embodiments, an antigen binding domain is murine, chimeric, human, or humanized.

In certain embodiments, a CAR used in a cellular immunotherapy composition described herein is engineered to target a pathogen specific antigen, an autoimmune disease associated antigen, or a tumor associated antigen. Examples of pathogen specific antigens include HIV antigens, HCV antigens, HBV antigens, CMV antigens, EBV antigens, parasitic antigens, and bacterial antigens. A target tumor associated antigen may be any antigen of clinical interest against which it would be desirable to trigger a cell mediated immune response that results in tumor killing. Non-limiting examples of tumor associated antigens that may be targeted by a CAR includes CD19, CD20, CD22, CD23, CD24, CD37, CD30, CD38, CD123, CA125, ROR1, mesothelin, CD33, CD56, c-Met, PSMA, EGFR, EGFRvIII, GD-2, GD-3, HPV E6, HPV E7, L1CAM, MUC-1, MUC-16, FcRH5, WT1, HER2, folate receptor α, VEGF-α, VEGFR1, VEGFR2, IL-13Rα2, IL-11Rα, MAGE-A1, PSA, ephrin A2, ephrin B2, Lewis Y antigen, NKG2D ligands, NY-ESO-1, TAG-72, CEA or the like.

A CAR binding domain is optionally followed by an extracellular, non-signaling spacer or linker region, which, for example, can position the antigen binding domain away from the T cell surface to enable proper cell/cell contact, antigen binding and activation (Patel et al., Gene Therapy 6: 412-419, 1999). An extracellular spacer region of a CAR is generally located between a hydrophobic portion or transmembrane domain and the extracellular binding domain. Spacer region length may be varied to maximize tumor recognition based on the selected target molecule, selected binding epitope, or antigen binding domain size and affinity (see, e.g., Guest et al., J. Immunother. 28:203-11, 2005; PCT Publication No. WO 2014/031687). In certain embodiments, a spacer region is an immunoglobulin hinge region. An immunoglobulin hinge region may be a wild type immunoglobulin hinge region or an altered wild type immunoglobulin hinge region. An altered IgG4 hinge region is described in PCT Publication No. WO 2014/031687, which hinge region is incorporated herein by reference in its entirety. Other examples of hinge regions used in the CARs described herein include the hinge region present in the extracellular regions of type 1 membrane proteins, such as CD8a, CD4, CD28 and CD7, which may be wild-type or variants thereof. In certain embodiments, an extracellular spacer region comprises all or a portion of an Fc domain selected from: a CH1 domain, a CH2 domain, a CH3 domain, or combinations thereof (see, e.g., PCT Publication WO 2014/031687, which spacers are incorporated herein by reference in their entirety).

A hydrophobic portion or transmembrane domain is disposed between an extracellular antigen binding domain, or the extracellular spacer region if present, and the intracellular signaling component. A transmembrane domain is a hydrophobic alpha helix that transverses host T cell membrane. In certain embodiments, a transmembrane domain is selected from the same molecule from which the ITAM-containing T cell activating motif is derived (e.g., CDζ, FcRγ) or from another type I transmembrane protein, such as CD4, CD8, or CD28.

An intracellular signaling component refers to the portion of a chimeric antigen receptor that transduces a signal to the inside of the T cell in response to binding of the CAR to the target antigen, eliciting an effector function, e.g., activation, cytokine production, proliferation, persistence, cytotoxic activity, homing, entry into the microenvironment of a tumor, or any combination thereof In some embodiments, a full length intracellular signaling component may be used. An intracellular signaling component of a CAR may be linked directly to the carboxyl terminus of the transmembrane domain or may be separated from the transmembrane domain by a spacer, linker or one or more junction amino acids. In some embodiments, a truncated portion of an intracellular signaling component is used, provided that the truncated portion retains sufficient signal transduction activity. In further embodiments, an intracellular signaling component is a variant of an entire or truncated portion of an intracellular signaling component, provided that the variant retains sufficient signal transduction activity (i.e., is a functional variant).

More robust T cell activation generally involves two distinct signaling events: (1) an antigen-specific signal provided through a T cell receptor (TCR) complex, which promotes T cell activation, and (2) a non-antigen specific costimulatory signal provided by the interaction between or the ligation of costimulatory molecules expressed on an antigen presenting cell and a T cell. In certain embodiments, an intracellular activation domain comprises an ITAM-containing T cell activating motif. An ITAM-containing T cell activating motif used in CARs of the instant disclosure can be identical to or functional variants of an intracellular signaling domain or portion thereof of an immune cell receptor, or of a cell surface marker containing at least one ITAM. In general, the ITAM-containing T cell activating motif provides a T cell activation signal upon CAR engagement with its target antigen (e.g., antigen in the context of an HLA or MHC complex). Non-limiting examples of ITAM containing intracellular signaling domains that may be used in the CARs described herein include those present on CD3γ, CD3δ, CD3ϵ, CD3ζ, FcRγ, CD38, CD5, CD22, CD79a, CD79b and CD66d. In particular embodiments, an ITAM-containing T cell activating motif is a CD3ζ ITAM-containing T cell activating motif.

CARs of this disclosure generally have an intracellular signaling component that comprises an intracellular costimulatory domain, such as a single intracellular costimulatory domain or multiple intracellular signaling domains. In related embodiments, a CAR of this disclosure has an intracellular signaling component comprised of an intracellular activation domain and a single intracellular costimulatory domain. An intracellular costimulatory domain provides a second or costimulatory signal to further promote a signal, e.g., a T cell response, which can include activation, cytokine production, proliferation, differentiation, survival, cytotoxicity, or any combination thereof. Non-limiting examples of costimulatory molecules having an intracellular costimulatory domain useful in the instant disclosure include CD27, CD28, 4-1BB (CD137), ICOS (CD278), OX40 (CD134), CD30, CD4OL, LFA-1, CD2, CD7, LIGHT, NKG2C, GITR, or the like.

The CARs described herein are designed such that those CARs destined for CD4+ T cells have an intracellular costimulatory domain selected for its ability to enhance CD4+ effector T cell activity or function. In certain embodiments, an intracellular costimulatory domain of a CAR used to modify a CD4+ T cell promotes TH1 cytokine section; promotes TH2 cytokine secretion; is upregulated upon activation of naïve CD4+ T cells or helper T cells; promotes secretion of IL-2 when ligated on a cell endogenously expressing the costimulatory molecule having a similar or identical intracellular costimulatory domain as contained on the CAR, or combinations thereof. In certain embodiments, an intracellular costimulatory domain of a CAR used to modify a CD4+ T cell does not comprise an intracellular signaling domain present in a molecule that is a marker of or primarily endogenous to a memory-lineage CD8+ T cell, marker of cell persistence, or marker of cell survival. In certain embodiments, intracellular costimulatory domains for a CAR used to modify a CD4+ T cell may be selected from costimulatory signaling domains of CD28, members of the CD28 family, ICOS, and OX40, and functional variants thereof; in some embodiments, it is not a costimulatory domain of ICOS and/or a functional variant thereof. In other embodiments, an intracellular costimulatory domain of a CAR used to modify a CD8+ T cell comprises an intracellular signaling domain from a costimulatory molecule that: promotes survival or persistence of a CD8+ T cell when ligated on a CD8+ T cell that endogenously expresses the costimulatory molecule, and/or is upregulated upon activation of naïve CD8+ T cells or CTL cells. In certain embodiments, intracellular costimulatory domains for a CAR used to modify a CD8+ T cell may be selected from costimulatory domains of 4-1BB, CD4OL, CD27, OX40, costimulatory members of the TNFR family, NKG2C, and GITR, and functional variants thereof. In some embodiments, it is not derived from a 4-1BB or a functional variant thereof.

CD28 is a costimulatory molecule that is constitutively expressed on all human CD4+ T cells and about 50% of human CD8+ T cells (Linsley et al., 1993, Annu. Rev. Immunol. 11:191-212; June et al., 1990, Immunol. Today 11:211-16). The ligands for

CD28 are B7-1 (CD80) and B7-2 (CD86), which are expressed on a variety of antigen presenting cells (APCs). CD28 is an “early” costimulatory molecule that has been shown to synergize with the TCR to lower the threshold of T cell activation, which is not attainable by TCR ligation alone, leading to enhanced survival and increased cytokine production (e.g., IL-2) needed for clonal expansion and differentiation (Bour-Jordan et al., 2011, Immunol. Rev. 241:180-205).

Inducible costimulatory (ICOS), also known as CD278, is a member of the CD28 family of costimulatory molecules whose expression is induced during activation of CD4+ T cells (Hutloff et al., 1999, Nature 397:263-266; Mages et al., 2000, Eur. J. Immunol. 30:1040-1047). ICOS has been shown to augment proliferation of activated CD4+ T cells (Hutloff et al., supra). ICOS costimulation has been found to regulate the survival of protective effector memory CD4+ T cells (Moore et al., 2012, PLoS One 6:e16529). It was initially thought that ICOS costimulated for the differentiation of TH2 CD4+ T cells (Coyle et al., 2000, Immunity 13:95-105; McAdam et al., 2000, J. Immunol. 154:5035-5040). Subsequent evidence showed that ICOS costimulation is required for both TH1 and TH2 type responses (Gonzalo et al., 2001. Nat. Immunol. 2:597-604; Ozkaynak et al., 2001, Nat. Immunol. 2001, 2:591-596; Rottman et al., 2001, Nat. Immunol. 2001, 2: 605-611; Smith et al., 2003, J Immunol. 170:2310-2315; Smith et al., 2006, Vaccine 24:3035-3043; Vidric et al., 2006, Infect. Immun. 74:1050-1061; Kopf et al., 2000, J Exp. Med. 192:53-61). It is suggested that the level of ICOS expression is correlated with the cytokines produced: CD4+ T cells expressing high levels of ICOS predominantly secrete IL-10; CD4+ T cells expressing intermediate levels of ICOS synthesized TH2 cytokines; and CD4+ T cells expressing low levels of ICOS made early cytokines such as IL-2, IFN-γ or GM-CSF (Lohning et al., 2003, J. Exp. Med. 197:181-193).

OX40 (CD134) is expressed primarily on CD4+ T cells. Engagement of OX40 enhances proliferation, cytokine production, survival, and migration of CD4+ T cells (Gramaglia et al., 1998, J. Immunol. 161:6510-6517; Gramaglia et al., 2000, J. Immunol. 165:3043-3050) and promotes function and effects of CD8+ cells. Bansal-Pakala et al., J. Immunol. 2004, 172: 4821-4825; Croft et al., Immunol. Rev. 2009, 229:173-91.

CD27 (also known as TNFRSF7) is expressed by naïve CD8+ T cells. Its ligation by CD70 promotes in vitro proliferation of TCR-stimulated CD8+ T cells (Hintzen et al., 1995, J. Immunol. 154:2612-2623; Rowley et al., 2004, J. Immunol. 172:6039-6046). CD27 costimulation may also promote long-term survival of primed CD8+ T cells (Huang et al., 2006, J. Immunol. 176:7726-7735; Oschsenbein et al. 2004, J. Exp. Med. 200:1407-1417; Huang et. al., 2006, J. Immunol. 176:7726-7735).

4-1BB (CD137) is primarily expressed by activated CD8+ T cells. Binding of 4-1BB with its ligand 4-1BBL expressed by activated DCs, B cells, and macrophages promotes the upregulation of anti-apoptotic molecules Bcl2 and Bcl-xl and protects tumor antigen specific cells from activation-induced cell death, thereby enhancing CD8+ T cell survival (Watts, 2005, Annu. Rev. Immunol. 23:23-68; Hernandez-Chacon et al., 2011, J. Immunother. 34:236-250; Moran et al., 2013, Curr. Opin. Immunol. 25:230-237).

Another member of the TNFR superfamily, glucocorticoid-induced TNFR-related protein (GITR), is found on both regulatory T cells and CD8+ T cells. GITR signaling on CD8+ results in increased expression of anti-apoptotic Bcl-xl and preferentially enhances CD8+ T cell expansion and survival (Snell et al., 2010, J. Immunol. 185:7223-34; Chen and Flies, 2013, Nat. Rev. Immunol. 13:227-242).

The inclusion of both an ITAM-containing T cell activating motif and an intracellular costimulatory domain within a CAR construct may enhance the efficacy, expansion, and survival of CAR modified T cells. Such CAR designs are referred to as second generation CARs, having an intracellular signaling domain providing a primary activating signal and an intracellular costimulatory domain. The ITAM-containing T cell activating motif and intracellular costimulatory domain may be linked in tandem and in any order.

In some embodiments, the CARs for use in adoptive cellular immunotherapy compositions provided herein do not include third generation CARs. Third generation CARs have at least two intracellular costimulatory domains combined with an intracellular signaling domain providing an activating signal.

Methods of making CARs are well known in the art and are described, for example, in U.S. Pat. No. 6,410,319; U.S. Pat. No. 7,446,191; U.S. Pat. Publication No. 2010/065818; U.S. Pat. No. 8,822,647; PCT Publication No. WO 2014/031687; U.S. Pat. No. 7,514,537; and Brentjens et al., 2007, Clin. Cancer Res. 13:5426, each of which is hereby incorporated by reference in its entirety.

The CAR constructs described herein are used to modify CD4+ and CD8+ T cells. In some embodiments, a CD4+ T cell is selected from the group consisting of naive CD4+ T cells, memory stem CD4+ T cells, central memory CD4+ T cells, effector memory CD4+ T cells, CD4+ T effector cells, bulk CD4+ T cells, and any combination thereof. A CD4+ T effector cell may refer to a TH1 CD4+ T effector cell or a TH2 CD4+ T effector cell. In some embodiments, CD4+ T cell comprises a population of CD4+ T cells that: are CD45RO0 negative and CD62L positive; are enriched for naive CD T cells, or that is a bulk population of CD4+ T cells. In more particular embodiments, a CD4+ T cell is a naive CD4+ T cell, wherein the naive CD4+ T cell comprises a CD45RO, CD45RA+, CD62L+, CD4+ T cell.

In some embodiments, the CD8+ T cell is selected from the group consisting of naive CD8+ T cells, memory stem CD8+ T cells, central memory CD8+ T cells, effector memory CD8+ T cells, effector CD8+ T cells, bulk CD8+ T cells, and any combination thereof. In some embodiments, the CD8+ T cell comprises a population of CD8+ T cells that are CD62L positive or are enriched for CD62L positive CD8+ T cells or central memory CD8+ T cells. In more particular embodiments, a CD8+ T cell is a central memory CD8+ T cell wherein the central memory CD8+ T cell comprises a CD45RO+, CD62L+, CD8+ T cell. In yet other embodiments, the CD8+ T cell is a central memory CD8+ T cell and the CD4+ T cell is a naive CD4+ T cell.

### Selection and Sorting of T Cell Populations

Prior to expansion and genetic modification of the T cells with a CAR construct, a source of T cells is obtained from subject (e.g., peripheral blood mononuclear cells, bone marrow, lymph node tissue, cord blood, thymus tissue, tissue from a site of infection, ascites, pleural effusion, spleen tissue), from which T cells are isolated using methods known in the art. Specific T cell subsets can be collected in accordance with known techniques and enriched or depleted by known techniques, such as affinity binding to antibodies, flow cytometry and/or immunomagnetic selection. After enrichment and/or depletion steps and introduction of a CAR, in vitro expansion of the desired modified T cells can be carried out in accordance with known techniques (including those described in U.S. Pat. No. 6,040,177), or variations thereof that will be apparent to those skilled in the art.

For example, a desired T cell population or subpopulation may be expanded by adding an initial T cell population to a culture medium in vitro, and then adding to the culture medium feeder cells, such as non-dividing peripheral blood mononuclear cells (PBMCs), (e.g., such that the resulting population of cells contains at least about 5, 10, 20, or 40 or more PBMC feeder cells for each T cell in the initial population to be expanded); and incubating the culture (e.g. for a time sufficient to expand the numbers of T cells). The non-dividing feeder cells can comprise gamma-irradiated PBMC feeder cells. In some embodiments, the PBMCs are irradiated with gamma rays in the range of about 3000 to 3600 rads. The order of addition of the T cells and feeder cells to the culture media can be reversed if desired. The culture can typically be incubated under conditions of temperature and the like that are suitable for the growth of T cells. For the growth of human T lymphocytes, for example, the temperature will generally be at least about 25° C., preferably at least about 30° C., more preferably about 37° C.

The T cells expanded include CAR-modified cytotoxic T lymphocytes (CTL) and CAR-modified helper T lymphocytes that are specific for an antigen present on a human tumor or a pathogen.

Optionally, the expansion method may further comprise the step of adding non-dividing EBV-transformed lymphoblastoid cells (LCL) as feeder cells. LCL can be irradiated with gamma rays in the range of about 6000 to 10,000 rads. The LCL feeder cells may be provided in any suitable amount, such as a ratio of LCL feeder cells to initial T lymphocytes of at least about 10:1.

Optionally, the expansion method may further comprise the step of adding anti-CD3 monoclonal antibody to the culture medium (e.g., at a concentration of at least about 0.5 ng/ml). Optionally, the expansion method may further comprise the step of adding IL-2 and/or IL-15 to the culture medium (e.g., wherein the concentration of IL-2 is at least about 10 units/ml).

After isolation of T lymphocytes, both CD8+ cytotoxic and CD4+ helper T lymphocytes can be sorted into naive, memory, and effector T cell subpopulations before genetically modifying with a CAR and expanding.

Whether a T cell or T cell population is positive for a particular cell surface marker can be determined by flow cytometry using staining with a specific antibody for the surface marker and an isotype matched control antibody. A cell population “negative” for a marker refers to the absence of significant staining of the cell population with the specific antibody above an isotype control, and “positive” refers to uniform staining of the cell population above the levels found on an isotype control. In some embodiments, a decrease in expression of one or more markers refers to a loss of 1 log10 in the mean fluorescence intensity and/or a percentage decrease of T cells that exhibit the marker of at least 20% of the cells, 25% of the cells, 30% of the cells, 35% of the cells, 40% of the cells, 45% of the cells, 50% of the cells, 55% of the cells, 60% of the cells, 65% of the cells, 70% of the cells, 75% of the cells, 80% of the cells, 85% of the cells, 90% of the cell, 95% of the cells, and 100% of the cells and any % between 20% and 100% when compared to a reference T cell population. In some embodiments, a T cell population positive for of one or markers refers to a percentage of cells that exhibit the marker, which may be at least 50% of the cells, 55% of the cells, 60% of the cells, 65% of the cells, 70% of the cells, 75% of the cells, 80% of the cells, 85% of the cells, 90% of the cell, 95% of the cells, and 100% of the cells and any % between 50% and 100% when compared to a reference T cell population.

Immunomagnetic selection methods may also be used to purify T cell subpopulations using commercially available clinical grade antibody bead conjugates using the CliniMACS device (see, e.g., Terakura et al., 2012, Blood 119:72-82; Wang et al., 2012, J. Immunother. 35:689-701). For example, to isolate human CD8+ T_{CM} cells, CD4+, CD14+ and CD45RA+ cells are removed from peripheral blood mononuclear cells by depletion with antibody conjugated paramagnetic beads, and then the CD62L+ fraction from the remaining cells is positively selected with an anti-CD62L labeled bead to enrich for the CD45RO+, CD62L+, CD8+ T_{CM} subpopulation. The enriched CD8+ T_{CM} subpopulation can be activated with anti-CD3/CD28 beads or with antigen, modified with tumor-specific CAR using retroviral or lentiviral vectors, and expanded for use in cellular immunotherapy (see, e.g., Terakura et al., supra; Wang et al., supra).

Alternatively, T cell subsets may be selected using low-affinity Fab fragments fused to Strep-tag II. The Fab monomers do not have sufficient binding affinity for stable binding to the target antigen on the cell surface. However, when multimerized on a StrepTactin bead, these reagents stably bind the target cell and enable selection based on cell surface marker specificity. The Fab multimer binding can be rapidly reversed by the addition of excess D-biotin, which has a higher affinity for StrepTactin and disrupts the binding between the Strep-tag on the Fab-fragment and the Strep-Tactin “backbone.” The Fab monomers cannot maintain stable binding to the cell. This “Fab-Streptamers” technology allows for serial positive enrichment of T cells based on multiple cell surface markers and can be used to select any desired T cell subset (see, e.g., Siemberger et al., PloS One 7:e35798, 2012).

Bulk CD8+ T cells can be obtained by using standard methods. In some embodiments, bulk CD8+ T cells are further sorted into naïve, central memory, and effector T cells by identifying certain cell surface markers that are associated with each of those types of CD8+ T cells. In certain embodiments, memory T cells are present in both CD62L+and CD62L− subsets of CD8+ peripheral blood lymphocytes. For example, PBMCs can be sorted into CD62L−CD8+ and CD62L+CD8+ fractions after staining with anti-CD8 and anti-CD62L antibodies. In some embodiments, the expression of phenotypic markers of CD8+ central memory T cells include CD45RO, CD62L, CCR7, CD28, CD3, and CD127 and are negative for granzyme B. In some embodiments, central memory T cells are CD45RO+, CD62L+, CD8+ T cells. In some embodiments, CD8+ effector T cells are negative for or have reduced expression of CD62L, CCR7, CD28 and CD127, and are positive for or have increased expression of granzyme B and perforin, as compared to CD8+ central memory T cells. In some embodiments, naïve CD8+ T cells are characterized by the expression of phenotypic markers of naïve T cells including CD62L, CCR7, CD28, CD3, CD127, and CD45RA.

Bulk CD4+ lymphocytes can be obtained by standard methods. In some embodiments, bulk CD4+ T cells are further sorted into naïve, central memory, and effector cells by identifying cell populations that have certain cell surface markers. In some embodiments, naïve CD4+ T lymphocytes are CD45RO−, CD45RA+, CD62L+, CD4+ T cell. In some embodiments, central memory CD4+ cells are CD62L positive and CD45RO positive. In some embodiments, effector CD4+ cells are CD62L and CD45RO negative or have reduced expression of CD62L and CD45RO as compared to central memory CD4+ cells.

Populations of CD4+ and CD8+ having TCRs that are antigen specific can be obtained by stimulating naïve or antigen-specific T lymphocytes with antigen. For example, T cell clones having antigen-specific TCRs can be generated against, for example, Cytomegalovirus antigens by isolating T cells from infected subjects and stimulating the cells in vitro with the same antigen. Naïve T cells may also be used by exposing them to peptide antigens presented in the context of an antigen presenting cell or a peptide-MHC complex. Any number of antigens from tumor cells, cancer cells, or pathogenic agents may be utilized. Examples of such antigens include HIV antigens, HCV antigens, HBV antigens, CMV antigens, EBV antigens, parasitic antigens, and tumor antigens, such as orphan tyrosine kinase receptor ROR1, EGFR, EGFRvIII, GD2, GD3, HPV E6, HPV E7, Her2, L1-CAM, Lewis A, Lewis Y, MUC1, MUC16, PSMA, CD19, CD20, CD22, CD56, CD23, CD24, CD37, CD30, CD33, CD38, CD56, CD123, CA125, c-MET, FcRH5, WT1, folate receptor α, VEGF-α, VEGFR1, VEGFR2, IL-13Rα2, IL-11Rα, MAGE-AL PSA, ephrin A2, ephrin B2, NKG2D ligands, NY-ESO-1, TAG-72, mesothelin, CEA or the like. Such T cells having antigen-specific TCRs may be further modified to contain a CAR as described herein, wherein the CAR is specific for the same antigen, specific for a different epitope on the same antigen, or specific for a different antigen. In any of these embodiments, the CD4+ T cells and the CD8+ T cells will contain different CARs, and in particular the intracellular signaling components of the CARs will be distinct.

Methods of preparing and modifying T cells to express CARs, confirming CAR modified T cell activity, expanding CAR modified T cell populations are known in the art and are described, for example, in Hollyman et al., 2009, J. Immunother. 32:169-180; PCT Publication No. WO 2012/079000; U.S. Pat. No. 8,802,374; Brentjens et al., Blood 118:4817-4828, 2011; U.S. Patent Publication No. U.S. 2014/0271635, the methods from each of which are incorporated by reference in its entirety.

### Adoptive Cellular Immunotherapy Compositions

In one aspect, present disclosure provides for an adoptive cellular immunotherapy composition comprising a genetically modified CD4+ T cell comprising a first chimeric antigen receptor (CAR), which first CAR specifically binds to an antigen and contains a first intracellular costimulatory domain; and a CD8+ T cell comprising a second CAR, which second CAR specifically binds to the antigen and contains a second intracellular costimulatory domain, which is distinct from the first intracellular costimulatory domain, wherein the CD4+ T cell does not contain the second CAR and/or does not contain a CAR comprising the second intracellular domain and/or wherein the composition does not contain any CD4+ T cell containing the second CAR or any CAR with the second intracellular domain; and/or wherein the CD8+ T cell does not contain the first CAR and/or does not contain a CAR comprising the first intracellular costimulatory domain and/or wherein the composition does not contain any CD8+ T cell containing the first CAR or any CAR with the first intracellular costimulatory domain. Both the first CAR and the second CAR bind to the same antigen. With the exception of the intracellular costimulatory domain, the first CAR and the second CAR may be identical or different with respect to the antigen binding domain, the optional spacer domain, the transmembrane domain, the ITAM-containing T cell activating motif, and any combination thereof.

In certain embodiments of the adoptive cellular immunotherapy composition, wherein in a co-culture of the CD4+ T cell and CD8+ T cell in vitro in the presence of the antigen: the level of a secreted Th1 cytokine produced and/or the degree of proliferation of the CD8+ T cell is greater as compared to a culture of the CD8+ T cell in the absence of the CD4+ T cell under the same conditions; and/or the level of a secreted Th1 cytokine produced and/or the degree of proliferation of the CD8+ T cell is greater as compared to a culture of the CD8+ T cell in the presence of a CD4+ T cell comprising a CAR that specifically binds to the antigen and contains the second intracellular costimulatory domain and/or does not contain the first intracellular costimulatory domain, under the same conditions. In certain embodiments of the adoptive cellular immunotherapy composition, a co-culture of the CD4+ T cell and CD8+ T cell in vitro, in the presence of the antigen, results in a greater level of a secreted TH1 cytokine and/or a greater degree of proliferation of the CD8+ T cell as compared to a culture of the CD8+ T cell: in the absence of the CD4+ T cell under the same conditions; and/or in the presence of a CD4+ T cell comprising a CAR containing the second intracellular costimulatory domain under the same conditions; and/or in the presence of a CD4+ T cell comprising a CAR lacking the first intracellular costimulatory domain under the same conditions. Co-culture of the CD4+ T cell and CD8+ T cell in vitro in the presence of the antigen may mean that the antigen (peptide) is presented to the CD4+ T cell and CD8+ T cell in the context of an antigen presenting cell (expressed on the surface of the antigen presenting cell) or in the context of a peptide-MHC complex. For presentation of a peptide antigen to a CD4+ T cell, the peptide-MHC complex comprises a MHC Class II molecule. For presentation of a peptide antigen to a CD8+ T cell, the peptide-MHC complex comprises a MHC Class I molecule.

In further embodiments, the secreted Th1 cytokine is selected from IL-2, IFN-γ, TNF-α, TNF-β, GM-CSF, or any combination thereof. Methods of measuring cytokine production are known in the art and include for example, measuring mRNA expression (e.g., qPCR, microarray) and measuring cytokine protein levels (e.g., multiplexed immunobead cytokine profiling, ELISA, and intracellular cytokine flow cytometry).

In further embodiments, the degree of CD8+ T cell proliferation is at least 5%, 10%, 20%, 30%, 40%, 50%, 60% 70%, 80%, 90%, 100%, 150%, 200%, 300%, 400%, 500%, 600%, 700%, 800%, or 900% greater. Methods of measuring T cell proliferation are known in the art and include for example, T cell quantitation by flow cytometry, T cell proliferation assays based on radioactive thymidine incorporation, and membrane label dilution assays.

In any of the preceding adoptive cellular immunotherapy embodiments, the first intracellular costimulatory domain may comprise an intracellular signaling domain of an endogenous costimulatory molecule expressed in helper T cells and/or a functional variant of said intracellular signaling domain. Non-limiting examples of a costimulatory molecule that is endogenously present in a helper T cell include CD28, ICOS, and OX40, and functional variants thereof

In any of the preceding adoptive cellular immunotherapy embodiments, the first intracellular costimulatory domain may comprise an intracellular signaling domain that is present in an endogenous costimulatory molecule that is expressed in helper T cells and promotes the secretion of a TH1 cytokine or differentiation into a TH1 phenotype, or a functional variant of said intracellular signaling domain. In any of the preceding adoptive cellular immunotherapy embodiments, the first intracellular costimulatory domain comprises an intracellular signaling domain present in an endogenous costimulatory molecule that is expressed in helper T cells and promotes the secretion of a TH2 cytokine or differentiation into a TH2 phenotype, or a functional variant of said intracellular signaling domain.

In further embodiments, the secreted TH1 cytokine is IL-2, IFN-γ, TNF-α, TNF-β, GM-CSF, or any combination thereof. In yet further embodiments, the secreted TH2 cytokine is IL-4, IL-5, IL-6, IL-9, IL-10, IL-13, IL-17E (IL-25), or any combination thereof.

In certain embodiments, the first intracellular costimulatory domain comprises an intracellular signaling domain present in an endogenous costimulatory molecule selected from the group consisting of CD28 family members, a costimulatory molecule upregulated upon activation of naïve CD4+ T cells, and a costimulatory molecule capable of promoting IL-2 secretion upon ligation in a cell endogenously expressing the costimulatory molecule, and functional variants thereof. The first intracellular costimulatory domain can be a domain present in an ICOS, a CD28, or a OX40, or a functional variant thereof In some embodiments, the first intracellular costimulatory domain is the domain present in an ICOS or a functional variant thereof. In some embodiments, the first intracellular costimulatory domain is the domain present in a CD28 or a functional variant thereof. In some embodiments, the first intracellular costimulatory domain is the domain present in an OX40 or a function variant thereof.

In any of the preceding adoptive cellular immunotherapy embodiments, the second intracellular costimulatory domain is a domain present in an endogous molecule that promotes survival or persistence when ligated on a CD8+ T cell endogenously expressing the molecule and/or that is upregulated upon activation of naïve CD8+ cells, or a functional variant thereof.

In certain embodiments, the second intracellular costimulatory domain is a domain present in an endogenous molecule that is not CD28 and/or that is a member of a TNFR family, or is a functional variant thereof. In some embodiments, the second intracellular costimulatory domain is a domain present in a CD40L, a 4-1BB, a CD27, an OX40, an NKG2C, or a GITR, or a functional variant thereof. In some embodiments, the second intracellular costimulatory domain is a domain present in a CD40L, a CD27, an NKG2C, or a GITR, or a functional variant thereof. In some embodiments, the second intracellular costimulatory domain is a domain present in a CD40L or a functional variant thereof. In some embodiments, the second intracellular costimulatory domain is a domain present in a CD27 or a function variant or portion thereof. In some embodiments, the second intracellular costimulatory domain is a domain present in a OX40 or a functional variant thereof In some embodiments, the second intracellular costimulatory domain is a domain present in a GITR or a functional variant thereof.

In certain embodiments, the first intracellular costimulatory domain does not comprise a costimulatory signaling domain derived from or present in an endogenous ICOS and/or wherein the second intracellular costimulatory domain does not comprise a costimulatory signaling domain derived from or present in an endogenous CD28.

In any of the preceding cellular immunotherapy embodiments, the first CAR and the second CAR may comprise the same antigen-binding domain and/or the same variable heavy chain domain and/or the same variable light chain domain, which binds to the antigen, optionally to the same or different epitope thereof or comprises the same transmembrane domain, the same spacer domain, and/or the same ITAM-containing T cell activating motif. By way of example, wherein the antigen binding domain is a scFv, the first CAR and second CAR may comprise the same scFvs. In another example, wherein the antigen binding domain is a variable heavy chain domain, the first

CAR and second CAR may comprise the same variable heavy chain domain. In yet another example, wherein the first CAR and the second CAR comprise different scFv antigen binding domains, each scFv may have the same variable heavy chain domain or the same variable light chain domain.

In any of the preceding adoptive cellular immunotherapy embodiments, the first costimulatory domain may (i) comprise an intracellular signaling domain from an endogenous costimulatory molecule that (a) promotes secretion of a TH1 cytokine or TH1 differentiation, (b) be upregulated upon activation of naive CD4+ T cells or helper T cells; and/or (c) promote secretion of IL-2 when ligated on a cell endogenously expressing the costimulatory molecule; and/or (ii) not comprise an intracellular signaling domain present in a molecule that is a marker of a memory-lineage CD8+ T cell, marker of cell persistence, or marker of cell survival; and/or the second costimulatory domain (i) comprises an intracellular signaling domain from an endogenous costimulatory molecule that (a) promotes survival or persistence of a CD8+ T cell when ligated on a CD8+ cell endogenously expressing the costimulatory molecule, and/or (b) is upregulated upon activation of naive CD8+ T or CTL cells.

In any of the preceding adoptive cellular immunotherapy embodiments, the first CAR does not comprise an intracellular signaling domain from more than one costimulatory molecule and/or does not comprise an intracellular costimulatory domain other than the first intracellular costimulatory domain, and/or is not a third generation CAR; and/or the second CAR does not comprise an intracellular signaling domain from more than one costimulatory molecule and/or does not comprise an intracellular costimulatory domain other than the second intracellular costimulatory domain, and/or is not a third generation CAR.

In any of the preceding adoptive cellular immunotherapy embodiments, the first intracellular costimulatory domain may comprise an intracellular signaling domain from a molecule selected from CD28, OX40, members of the CD28 family, and ICOS and/or the second intracellular costimulatory domain may comprise an intracellular signaling domain from a molecule selected from 4-1BB, GITR, NKG2C, OX40, CD40L, costimulatory members of the TNFR family, and CD27. In certain embodiments, the first intracellular costimulatory domain comprises an intracellular signaling domain from a molecule selected from CD28 and ICOS and/or wherein the second intracellular costimulatory domain comprises an intracellular signaling domain from a molecule selected from 4-1BB and CD27. In certain embodiments, the first intracellular costimulatory domain comprises an intracellular signaling domain from a CD28 molecule and the second intracellular costimulatory domain comprises an intracellular signaling domain from a molecule selected from 4-1BB and CD27. In certain embodiments, the first intracellular costimulatory domain comprises an intracellular signaling domain from a molecule selected from CD28 and ICOS and the second intracellular costimulatory domain comprises an intracellular signaling domain from a CD27 molecule.

In any of the preceding adoptive cellular immunotherapy embodiments, the first and/or second CAR comprises a single-chain antibody fragment (scFv) which binds to the antigen. In some embodiments, the single-chain antibody fragment is chimeric, human, or humanized.

In any of the preceding adoptive cellular immunotherapy embodiments, the CD4+ T cell may comprise a population of CD4+ cells: that are CD45RO negative and CD62L positive, that are enriched for naive CD4+ T cells, or is a bulk population of CD4+ T cells; and/or wherein the CD8+ T cell comprises a population of CD8+ cells: that are CD62L positive or that are enriched for CD62L positive CD8+ T cells or central memory CD8+ T cells.

The present disclosure also provides a composition or combination comprising one or more nucleic acids encoding the first CAR and the second CAR of any of the preceding adoptive immunotherapy compositions. In certain embodiments, the one or more nucleic acids comprises a nucleic acid encoding the first CAR and a nucleic acid encoding the second CAR. In certain embodiments, each of the nucleic acid encoding the first CAR and the nucleic acid encoding the second CAR, individually, is comprised within a vector, optionally on the same vector or different vectors.

The present disclosure also provides a vector comprising a nucleic acid encoding the first CAR and a nucleic acid encoding the second CAR according to any of the preceding adoptive immunotherapy compositions.

### Methods of Treatment

The present disclosure provides methods of performing cellular immunotherapy in a subject having a disease, condition, or disorder comprising: administering any of the adoptive cellular immunotherapy compositions described herein to the subject.

Another embodiment provides a method of performing cellular immunotherapy in a subject having a disease, condition, or disorder comprising analyzing a biological sample of the subject for the presence of an antigen associated with the disease or disorder and administering an adoptive cellular immunotherapy composition described herein, wherein the chimeric antigen receptor specifically binds to the antigen. In some embodiments, the antigen associated with the disease or disorder is a tumor-associated antigen. Subjects that can be treated by the present invention are, in general, human and other primate subjects, such as monkeys and apes for veterinary medicine purposes. The subjects can be male or female and can be any suitable age, including infant, juvenile, adolescent, adult, and geriatric subjects.

Subjects that can be treated include subjects afflicted with cancer, including solid tumors, a hematological malignancy, melanoma, non-small cell lung cancer, renal cell carcinoma, renal cancer, a hematological cancer, prostate cancer, castration-resistant prostate cancer, colon cancer, rectal cancer, gastric cancer, esophageal cancer, bladder cancer, head and neck cancer, thyroid cancer, breast cancer, triple-negative breast cancer, ovarian cancer, cervical cancer, lung cancer, urothelial cancer, pancreatic cancer, glioblastoma, hepatocellular cancer, myeloma, multiple myeloma, leukemia, Hodgkin's lymphoma, non-Hodgkin's lymphoma, myelodysplastic syndrome, brain cancer, CNS cancer, malignant glioma, bone cancer, or any combination thereof.

Subjects that can be treated also include subjects afflicted with, or at risk of developing, an infectious disease, including viral, retroviral, bacterial, and protozoal infections. In certain embodiments, subjects are immune compromised or immunodeficient and afflicted with a viral infection, such as a Cytomegalovirus (CMV), Epstein-Barr virus (EBV), adenovirus, HIV, or BK polyomavirus infection. For example, an immune compromised or immunodeficient subject may be a transplant patient, a cancer patient, a patient having a congenital disorder, or the like.

Adoptive cellular immunotherapy compositions described herein are administered to subjects in accordance with known techniques, or variations thereof that will be apparent to those skilled in the art.

In some embodiments, the cells are prepared by harvesting the cells (from a biological sample, tissue or culture medium), washing, concentrating, and formulating in a medium and container system suitable for administration (a “pharmaceutically acceptable” carrier) in a treatment-effective amount. Suitable infusion media can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin or other human serum components.

“Effective amount” or “therapeutically effective amount” refers to that amount of a composition described herein which, when administered to a mammal (e.g., human), is sufficient to aid in treating a disease. The amount of a composition that constitutes a “therapeutically effective amount” will vary depending on the cell preparations, the condition and its severity, the manner of administration, and the age of the mammal to be treated, but can be determined routinely by one of ordinary skill in the art having regard to his own knowledge and to this disclosure. When referring to an individual active ingredient or composition, administered alone, a therapeutically effective dose refers to that ingredient or composition alone. When referring to a combination, a therapeutically effective dose refers to combined amounts of the active ingredients, compositions or both that result in the therapeutic effect, whether administered serially, concurrently or simultaneously.

A treatment effective amount of cells in a composition is at least one cell (for example, one CAR modified CD8+ T cell subpopulation; one CAR modified CD4+ T cell subpopulation) or is more typically greater than 10² cells, for example, up to 10⁶, up to 10⁷, up to 10⁸ cells, up to 10⁹ cells or more than 10¹⁰ cells. In certain embodiments, the cells are administered in a range from about 10⁶ to about 10¹⁰ cells/m², preferably in a range of about 10⁷ to about 10⁹ cells/m². The number of cells will depend upon the ultimate use for which the composition is intended as well the type of cells included therein. For example, cells modified to contain a CAR specific for a particular antigen will comprise a cell population containing at least 30%, 35%, 40%, 45%, 50%, 55%, 60%, 65%, 70%, 75%, 80%, 85%, 90%, 95% or more of such cells. For uses provided herein, the cells are generally in a volume of a liter or less, 500 mls or less, 250 mls or less, or 100 mls or less. Hence the density of the desired cells is typically greater than 10⁴ cells/ml and generally is greater than 10⁷ cells/ml, generally 10⁸ cells/ml or greater.

The cells may be administered as a single infusion or in multiple infusions over a range of time. A clinically relevant number of immune cells can be apportioned into multiple infusions that cumulatively equal or exceed 10⁶, 10⁷, 10⁸, 10⁹, 10¹⁰ or 10¹¹ cells. In certain embodiments, a composition of modified CD4+ T cells and a composition of modified CD8+ T cells are both administered, which administration may be simultaneous, concurrent or sequential.

In some embodiments, the lymphocytes of this disclosure may be used to confer immunity to individuals. By “immunity” is meant a lessening of one or more physical symptoms associated with a response to infection by a pathogen, or to a tumor, to which the lymphocyte response is directed. The amount of cells administered is usually in the range present in normal individuals with immunity to the pathogen. Since different individuals are expected to vary in responsiveness, the type and amount of cells infused, as well as the number of infusions and the time range over which multiple infusions are given are determined by the attending physician, and can be determined by routine examination. For example, the generation of sufficient levels of CAR modified T lymphocytes (including CD8+ T cells and/or CD4+ T cells) is readily achievable using a version of the rapid expansion method as described in U.S. Pat. No. 6,040,177.

In some embodiments, a composition as described herein is administered intravenously, intraperitoneally, intratumorly, into the bone marrow, into the lymph node, and/or into cerebrospinal fluid. In some embodiments, chimeric antigen receptor engineered compositions are delivered to the site of the tumor.

In some embodiments, the compositions as described herein are administered with chemotherapeutic agents and/or immune modulators (e.g., immunosuppressants, inhibitors of immunosuppression components such as immune checkpoint inhibitors). Immune checkpoint inhibitors include inhibitors of CTLA-4, A2AR, B7-H3, B7-H4, BTLA, HVEM, GALS, IDO, KIR, LAG-3, PD-1, PD-L1, PD-L2, Tim-3, VISTA, TIGIT, LAIR1, CD160, 2B4, TGFR beta, CEACAM-1, CEACAM-3, CEACAM-5, CD244, or any combination thereof. An inhibitor of an immune checkpoint molecule can be an antibody or antigen binding fragment thereof, a fusion protein, a small molecule, an RNAi molecule, (e.g., siRNA, shRNA, miRNA), a ribozyme, an aptamer, or an antisense oligonucleotide. A chemotherapeutic can be a B-Raf inhibitor, a MEK inhibitor, a VEGF inhibitor, a VEGFR inhibitor, a tyrosine kinase inhibitor, an anti-mitotic agent, or any combination thereof. In an embodiment, the chemotherapeutic is vemurafenib, dabrafenib, trametinib, cobimetinib, sunitinib, erlotinib, paclitaxel, docetaxel, or any combination thereof. In an embodiment, a patient is first treated with a chemotherapeutic agent that inhibits or destroys other immune cells followed by the compositions described herein. In some cases, chemotherapy may be avoided entirely.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including but not limited to U.S. Provisional Patent Application No. 62/168,675 filed on May 29, 2015, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. An adoptive cellular immunotherapy composition, comprising:
(a) a CD4+ T cell comprising a first chimeric antigen receptor (CAR), which first CAR specifically binds to an antigen and contains a first intracellular costimulatory domain; and
(b) a CD8+ T cell comprising a second CAR, which second CAR specifically binds to the antigen and contains a second intracellular costimulatory domain, which is distinct from the first intracellular costimulatory domain,
wherein the CD4+ T cell does not contain the second CAR and/or does not contain a CAR comprising the second intracellular costimulatory domain and/or wherein the composition does not contain any CD4+ T cell containing the second CAR or any CAR with the second intracellular costimulatory domain; and/or
wherein the CD8+ T cell does not contain the first CAR and/or does not contain a CAR comprising the first intracellular costimulatory domain and/or wherein the composition does not contain any CD8+ T cell containing the first CAR or any CAR with the first intracellular costimulatory domain.

2. The adoptive cellular immunotherapy composition of claim 1, wherein a co-culture of the CD4+ T cell and CD8+ T cell in vitro, in the presence of the antigen, results in a greater level of a secreted TH1 cytokine and/or a greater degree of proliferation of the CD8+ T cell as compared to a culture of the CD8+ T cell:
(a) in the absence of the CD4+ T cell under the same conditions; and/or
(b) in the presence of a CD4+ T cell comprising a CAR containing the second intracellular costimulatory domain under the same conditions; and/or
(c) in the presence of a CD4+ T cell comprising a CAR lacking the first intracellular costimulatory domain under the same conditions.

3. The adoptive cellular immunotherapy composition of claim 1 or 2, wherein the antigen is presented in a MHC-peptide complex or wherein the antigen is an antigen expressed on the surface of an antigen presenting cell.

4. The adoptive cellular immunotherapy composition of any of claims 1-3, wherein the first intracellular costimulatory domain comprises an intracellular signaling domain of an endogenous costimulatory molecule expressed in helper T cells, or a functional variant of said intracellular signaling domain.

5. The adoptive cellular immunotherapy composition of any of claims 1-4, wherein the first intracellular costimulatory domain comprises an intracellular signaling domain present in an endogenous costimulatory molecule that is expressed in helper T cells and that promotes the secretion of a TH1 cytokine or differentiation into a TH1 phenotype, or a functional variant of said intracellular signaling domain.

6. The adoptive cellular immunotherapy composition of any of claims 1-5, wherein the first intracellular costimulatory domain comprises an intracellular signaling domain present in an endogenous costimulatory molecule that is expressed in helper T cells and that promotes the secretion of a TH2 cytokine or differentiation into a TH2 phenotype, or a functional variant of such an intracellular signaling domain.

7. The adoptive cellular immunotherapy composition of any of claims 1-6, wherein the first intracellular costimulatory domain comprises an intracellular signaling domain present in an endogenous costimulatory molecule selected from the group consisting of CD28 family members, a costimulatory molecule upregulated upon activation of naïve CD4+ T cells, and a costimulatory molecule capable of promoting IL-2 secretion upon ligation in a cell endogenously expressing the costimulatory molecule, and functional variants thereof.

8. The adoptive cellular immunotherapy composition of any of claims 1-7, wherein the first intracellular costimulatory domain is a domain present in an ICOS, a CD28, or a OX40, or a functional variant thereof.

9. The adoptive immunotherapy composition of claim 8 wherein the first intracellular costimulatory domain is the domain present in an ICOS or a functional variant thereof.

10. The adoptive immunotherapy composition of claim 8, wherein the first intracellular costimulatory domain is the domain present in a CD28 or a functional variant thereof.

11. The adoptive immunotherapy composition of claim 8, wherein the first intracellular costimulatory domain is the domain present in an OX40 or a functional variant thereof.

12. The adoptive immunotherapy composition of any of claims 1-11, wherein the second intracellular costimulatory domain is a domain present in an endogenous molecule that promotes survival or persistence when ligated on a CD8+ T cell endogenously expressing the molecule and/or that is upregulated upon activation of naïve CD8+ cells, or a functional variant thereof

13. The adoptive immunotherapy composition of any of claims 1-12, wherein the second intracellular costimulatory domain is a domain present in an endogenous molecule that is not CD28 and/or that is a member of a TNFR family, or is a functional variant thereof.

14. The adoptive immunotherapy composition of any of claims 1-13, wherein the second intracellular costimulatory domain is a domain present in a CD40L, a 4-1BB, a CD27, an OX40, an NKG2C, or a GITR, or a functional variant thereof

15. The adoptive immunotherapy composition of any of claims 1-14, wherein the second intracellular costimulatory domain is a domain present in a CD40L, a CD27, an NKG2C, or a GITR, or a functional variant thereof.

16. The adoptive immunotherapy composition of any of claims 1-15, wherein the second intracellular costimulatory domain is a domain present in a CD40L or a functional variant thereof.

17. The adoptive immunotherapy composition of any of claims 1-16, wherein the second intracellular costimulatory domain is a domain present in a CD27 or a functional variant or portion thereof,

18. The adoptive immunotherapy composition of any of claims 1-17, wherein the second intracellular costimulatory domain is a domain present in an OX40 or a functional variant thereof.

19. The adoptive immunotherapy composition of any of claims 1-18, wherein the second intracellular costimulatory domain is a domain present in a GITR or a functional variant thereof.

20. The adoptive immunotherapy composition of any of claims 1-19, wherein the first intracellular costimulatory domain does not comprise a costimulatory signaling domain derived from or present in an endogenous ICOS and/or wherein the second intracellular costimulatory domain does not comprise a costimulatory signaling domain derived from or present in an endogenous CD28.

21. The adoptive cellular immunotherapy composition of any of claims 1-5, wherein the second intracellular costimulatory domain is a domain present in an immunotherapy composition of any of claims 1-20, wherein the first CAR and the second CAR comprise the same antigen-binding domain and/or the same variable heavy chain domain and/or the same variable light chain domain; or comprise the same transmembrane domain, the same spacer domain, and/or the same ITAM-containing T cell activating motif.

22. The adoptive cellular immunotherapy composition of any of claims 1-21, wherein:
the first costimulatory domain: (i) comprises an intracellular signaling domain from an endogenous costimulatory molecule that (a) promotes secretion of a TH1 cytokine or TH1 differentiation, (b) is upregulated upon activation of naive CD4+ T cells or helper T cells;
and/or (c) promotes secretion of IL-2 when ligated on a cell endogenously expressing the costimulatory molecule; and/or (ii) does not comprise an intracellular signaling domain present in a marker of a memory-lineage CD8+ T cell, marker of cell persistence, or marker of cell survival; and/or
the second costimulatory domain (i) comprises an intracellular signaling domain from an endogenous costimulatory molecule that (a) promotes survival or persistence of a CD8+ T cell when ligated on a CD8+ cell endogenously expressing the costimulatory molecule, and/or (b) is upregulated upon activation of naive CD8+ T cells or CTL cells.

23. The adoptive cellular immunotherapy composition of any of claims 1-22, wherein:
the first CAR does not comprise an intracellular signaling domain from more than one costimulatory molecule and/or does not comprise an intracellular costimulatory domain other than the first intracellular costimulatory domain, and/or is not a third generation CAR; and/or
the second CAR does not comprise an intracellular signaling domain from more than one costimulatory molecule and/or does not comprise an intracellular costimulatory domain other than the second intracellular costimulatory domain, and/or is not a third generation CAR.

24. The adoptive cellular immunotherapy composition of any of claims 1-23, wherein the first intracellular costimulatory domain comprises an intracellular signaling domain from a molecule selected from CD28 and ICOS and/or wherein the second intracellular costimulatory domain comprises an intracellular signaling domain from a molecule selected from 4-1BB and CD27.

25. The adoptive cellular immunotherapy composition of any of claims 1-24, wherein the first intracellular costimulatory domain comprises an intracellular signaling domain from a CD28 molecule and the second intracellular costimulatory domain comprises an intracellular signaling domain from a molecule selected from 4-1BB and CD27.

26. The adoptive cellular immunotherapy composition of any of claims 1-25, wherein the first intracellular costimulatory domain comprises an intracellular signaling domain of a molecule selected from CD28 and ICOS and the second intracellular costimulatory domain comprises an intracellular signaling domain from a CD27 molecule.

27. The adoptive cellular immunotherapy composition of any of claims 1-26, wherein an antigen binding domain of the first and/or second CAR comprises a single-chain antibody fragment (scFv).

28. The adoptive cellular immunotherapy composition of any of claims 1-27,
wherein the CD4+ T cell modified to contain the first CAR comprises a population of CD4+ cells that are CD45RO negative and CD62L positive, enriched for naive CD4+ T cells, or a bulk population of CD4+ T cells, and/or
wherein the CD8+ T cell modified to contain the second CAR comprises a population of CD8+ cells that are CD62L positive, enriched for CD62L positive CD8+ T cells, or central memory CD8+ T cells.

29. A composition or combination comprising one or more nucleic acids encoding the first CAR and the second CAR of the adoptive immunotherapy composition of any of claims 1-28.

30. The composition or combination of claim 29, wherein the one or more nucleic acids comprise a nucleic acid encoding the first CAR and a nucleic acid encoding the second CAR.

31. The composition or combination of claim 30, wherein each of the nucleic acid encoding the first CAR and the nucleic acid encoding the second CAR, individually, is comprised within a vector, optionally on the same vector or different vectors.

32. A vector comprising a nucleic acid encoding the first CAR and a nucleic acid encoding the second CAR according to the adoptive cellular immunotherapy composition of any of claims 1-28.

33. A kit comprising an adoptive cellular immunotherapy, comprising:
(a) a composition of modified CD4+ T cells comprising a first chimeric antigen receptor (CAR), the first CAR containing an extracellular antigen binding domain capable of specifically binding to an antigen and a first intracellular costimulatory domain; and
(b) a composition of modified CD8+ T cells comprising a second CAR, the second CAR containing an extracellular antigen binding domain capable of specifically binding to the antigen and a second intracellular costimulatory domain,
provided that the first and second intracellular costimulatory domains are distinct, and
wherein the CD4+ T cell composition does not contain the second CAR, does not contain a CAR comprising the second intracellular costimulatory domain, or both; and
wherein the CD8+ T cell composition does not contain the first CAR, does not contain a CAR comprising the first intracellular costimulatory domain, or both.

34. A method of treating cancer disease or condition in a subject, the method comprising administering to the subject an effective amount of an immunotherapy comprised of a composition of modified CD4+ T cells comprising a first chimeric antigen receptor (CAR) and a composition of modified CD8+ T cells comprising a second CAR according to any one of claims 1-28, wherein the disease or condition is optionally a cancer.

35. The method of claim 34, wherein the composition of modified CD4+ T cells and composition of modified CD8+ T cells are administered simultaneously, concurrently or sequentially.

36. The method of claim 34 or 35, wherein the cancer is a solid tumor, melanoma, non-small cell lung cancer, renal cell carcinoma, renal cancer, a hematological cancer, prostate cancer, castration-resistant prostate cancer, colon cancer, rectal cancer, gastric cancer, esophageal cancer, bladder cancer, head and neck cancer, thyroid cancer, breast cancer, triple-negative breast cancer, ovarian cancer, cervical cancer, lung cancer, urothelial cancer, pancreatic cancer, glioblastoma, hepatocellular cancer, myeloma, multiple myeloma, leukemia, Hodgkin's lymphoma, non-Hodgkin's lymphoma, myelodysplastic syndrome, brain cancer, CNS cancer, or malignant glioma.

37. The method of claim any of claims 34-36, wherein the immunotherapy further comprises administering a chemotherapeutic or an inhibitor of an immunosuppression component.

38. The method of claim 37, wherein the inhibitor of an immunosuppression component is an antibody or siRNA.

39. The method of claim 38, wherein the antibody or siRNA is specific for PD-1, PD-L1, PD-L2, LAG3, CTLA4, KIR, CD244, B7-H3, B7-H4, BTLA, HVEM, GALS, TIM3, A2aR, or any combination thereof

40. The method of claim 37, wherein the chemotherapeutic is a B-Raf inhibitor, a MEK inhibitor, a VEGF inhibitor, a VEGFR inhibitor, a tyrosine kinase inhibitor, an anti-mitotic agent, or any combination thereof

41. The method of claim 37, wherein the chemotherapeutic is vemurafenib, dabrafenib, trametinib, cobimetinib, sunitinib, erlotinib, paclitaxel, docetaxel, or any combination thereof.
